(19) United States
(12) Patent Application Publication
(10) Pub. No.: US 2024/0426800 A1
(43) Pub. Date: Dec. 26, 2024

(54) Remote Water Monitoring System

(71) Applicant: Mid-South Chemical Company, Inc., Lafayette, LA (US)

(72) Inventors: Kevin Ayers, Lafayette, LA (US); Will Breaux, Lafayette, LA (US)

(21) Appl. No.: 18/752,149

(22) Filed: Jun. 24, 2024

Related U.S. Application Data
(60) Provisional application No. 63/509,551, filed on Jun. 22, 2023.

(51) Int. Cl. G01N 33/18

(52) U.S. Cl. CPC G01N 33/18

(57) ABSTRACT

A remote water surveillance, or monitoring, system that can be used to recirculate and test stored water that is waiting to be distributed to areas affected by natural disasters or with poor water quality. A remote water monitoring system that is designed to track and report water quality levels in real time and allow a network of operators to monitor said water quality levels remotely. A remote water monitoring system that can ensure that potable water stays within the drinking water parameters and quality levels while waiting to be distributed.

### CROSS REFERENCES TO RELATED APPLICATION

Priority of U.S. provisional patent application Ser. No. 63/509,551, filed Jun. 22, 2023, incorporated herein by reference, is hereby claimed.

### STATEMENTS AS TO THE RIGHTS TO THE INVENTION MADE UNDER FEDERALLY SPONSORED RESEARCH AND DEVELOPMENT

None

### BACKGROUND OF THE INVENTION

### Field of the Invention

The present invention pertains to a remote water surveillance, or monitoring, system that can be used to recirculate and test stored water that is waiting to be distributed to areas affected by natural disasters or with poor water quality. More particularly, the present invention pertains to a water monitoring system that is designed to track and report water quality levels in real time and allow a network of operators to monitor said water quality levels remotely. Moreover, the system of the present invention can ensure that potable water stays within the drinking water parameters and quality levels while waiting to be distributed.

### Brief Description of the Prior Art

The National Institutes of Health (NIH) defines drinking-water supply surveillance as “the continuous and vigilant public health assessment and review of the safety and acceptability of drinking-water supplies.” However, the conventional surveillance systems in the drinking water industry typically operate with outdated equipment for treating and monitoring their drinking water quality. While some systems have monitoring equipment installed, it is often older and inaccurate, and other systems rely on an operator to take samples daily and to send those samples to a laboratory for evaluation. This process, even when done correctly, does not accurately capture how the quality of water can vary throughout the day and under different conditions. There have been many cases of operators failing to collect and submit these samples and falsifying reports. As such, there is a need for the remote water surveillance system of the present invention that offers a variety of benefits to smaller water systems over traditional monitoring with grab samples.

For example, the present invention allows a user to check for notable parameters of the water quality. Continuous monitoring allows for the early detection of contaminants such as heavy metals, and microbial pathogens. By promptly identifying these substances, water treatment facilities can take immediate action to mitigate risks to public health and prevent widespread contamination.

Additionally, the present invention ensures compliance with regulations. Regular monitoring ensures that drinking water meets regulatory standards set by authorities such as the Environmental Protection Agency (EPA) or the Louisiana Department of Health (LDH). By consistently maintaining compliance, water systems avoid costly fines and legal consequences while upholding their duty to provide safe drinking water to consumers.

The present invention further prevents waterborne diseases. Monitoring physical properties, such as turbidity, pH, and temperature, helps identify conditions conducive to the growth of harmful bacteria like E. coli and Legionella. Timely detection enables water treatment facilities to implement appropriate disinfection measures, thereby preventing waterborne diseases and outbreaks within communities.

Moreover, the present invention can optimize treatment processes. Continuous monitoring provides valuable data on the effectiveness of water treatment processes. By tracking parameters of the water quality in real-time, operators can adjust treatment methods accordingly, thereby ensuring efficient removal of contaminants and maintaining water quality standards without excessive use of chemicals.

As a result, the water surveillance system of the present invention can be used in disaster relief efforts to ensure that potable water is being delivered to end users. Further, the data collected and organized into daily reports will ensure that water quality is always within the parameters of the local drinking water standards. This system is especially useful in the disaster relief sector where water is often trucked in and staged in external storage tanks for relatively long periods of time. The water quality in these tanks can quickly turn when left stagnant and exposed to heat and sunlight, and the water delivered to end users is often well below the required chlorine residual level. When the chlorine residual level drops, the likelihood of bacteria and other biological growth increases substantially. Thus, the present invention can mitigate the risk of illness, eliminate waste caused by disposing of non-potable water, and provide much needed oversight in the drinking water industry.

### SUMMARY OF THE INVENTION

The present invention pertains to a remote water surveillance, or monitoring, system that can be used to recirculate and test stored water that is waiting to be distributed to areas affected by natural disasters. The system of the present invention can ensure that potable water stays within the drinking water parameters while waiting to be distributed by monitoring with a variety of different types of probes and using a chemical dosing pump that can adjust a variety of different residual levels of the water. This system of the present invention can have telemetry technology to allow an operator to monitor water quality remotely.

The present invention pertains to a water monitoring system that is designed to track and report water quality levels in real time and allow a network of operators to monitor said water quality levels remotely. The system of the present invention has the capability to monitor the quality of the water circulating through it as well as the ability to inject a variety of chemicals into said water in order to keep chlorine levels, PH levels, and any other monitored parameters in compliance with local standards.

The water monitoring system of the present invention allows for a network of water operators to be able to monitor stored potable water for approximately twenty-four (24) hours a day and ensure that water quality is always in compliance with state and local standards. This constant monitoring of free and total chlorine, conductivity, oxidation-reduction potential (ORP), temperature, and PH levels helps guarantee that the water is always safe. In addition, a plurality of sampling ports are available for certified operators to be able to take water samples from the system in order to be submitted to a testing lab as needed or as required by the end user.

The water monitoring system of the present invention has the ability to tie into existing water systems, facilities with downed water systems, neighborhoods, and temporary water staging and distribution systems for disaster relief applications. Additionally, the water monitoring system has the ability to tie into existing water systems for a period of time in order to create a data set illustrating water quality levels over multiple weeks. This will give the system a better picture of how the quality of their water varies with usage and other external parameters.

Additionally, the water monitoring system of the present invention further comprises an intake quality validation process that can analyze and contain a relatively small portion of water until it has been determined that the water quality meets the established parameters of the underlying application. If the water quality is confirmed to be of a satisfactory standard, the water will then be sent through the system to a storage tank, or other similar storage location. If the water quality is found to be contaminated, the water will be sent to a separate disposal line. The validation process is designed for remote applications and aims to quickly ensure that the water is of an acceptable quality without having to send a sample to a lab.

In a preferred embodiment, the water monitoring system of the present invention typically requires a steel Skid, a frame, and a plurality of fittings. Within the frame, the present invention further comprises a plurality of pumps, a plurality of valves, at least one probe or sensor, at least one flowmeter, and a plurality of hoses.

The water monitoring system of the present invention generally comprises multiple functions and applications, including, but not limited to, recirculation and monitoring, filling a storage tank from a truck, and filling a truck from a storage tank.

For example, when recirculating and monitoring stored water, the water from a storage tank is pumped into the system. The water passes through a three-way valve in a closed position, water then passes over a probe, through a 3-way closed valve, through a plurality of injection pumps, and out back to a tank. A programmable logic controller (PLC) communicates with a plurality of probes or sensors, including, but not limited to a multi-parameter sensor, or any other sensor. The multi-parameter sensors provide real time monitoring of a variety of parameters, including, but not limited to, free and total chlorine, conductivity and turbidity, pH, ORP and temperature, flow rate in gallons, pressure, and volume of water stored in the tank. Moreover, by way of illustration, but not limitation, additional parameters that could be monitored in the system include: chlorophyll, nitrogen, dissolved oxygen, arsenic, bromine, phosphorous, ammonia, total dissolved solids, total suspended solids, alkalinity, biochemical oxygen demand, chemical oxygen demand, and hardness. The multi-parameter sensors could also monitor for ATP (Adenosine triphosphate), which is an analysis of DNA in the sample to give data on the microbial presence which can indicate the potential for microorganisms to grow in the sample. The presence of ATP could trigger a grab sample to be collected if it is over a pre-determined threshold and sent to a lab for further analysis. Additionally, the sensors could also monitor for PFAS (per- and polyfluoroalkyl substances) in order to comply with new and future drinking water regulations.

The system of the present invention can use the multi-parameter sensors and the injection pumps to maintain continuous dosing to keep the water within the established parameters. Moreover, the water monitoring system is capable of monitoring said various parameters of stored water within the system in order to ensure that the potable water doesn't deteriorate and have to be discarded.

When filling a tank from a truck, a truck pulls up to the water monitoring system and a driver then connects a hose from the truck to a bulkhead connection on the system. The driver then presses a push button on an external panel of the unit. A pump on the intake line will then pump the water into the system, through the flowmeters that will record water quantity being taken in, through the probe to establish if quality is acceptable, and if so, then deposit the water into the tank.

In addition, when filling a tank from a truck, if a quality validation process is needed first, the source water is hooked up to the intake stream. The operator will then press a button to start the initial intake. The PLC will then activate a pump in order to take in a predetermined amount of water into an intake loop. The system will then recirculate the water within the intake loop until the various probes can relay their readings to the PLC. Once the PLC has received and determined if the water is of an acceptable quality, it will operate a three-way valve to release the acceptable water into the larger system and the intake process will continue. If the water is determined to be of unacceptable quality, an alarm will alert the operator, and then the unacceptable water will be diverted to a waste stream and then discarded.

Alternatively, when filling a truck from a tank, a driver pulls up to the water monitoring system and the driver will connect a hose to the unit, press the push button to fill their truck and then wait for the tanker truck to fill. The pump on the recirculation loop will continue to run, but after the probes, the 3-way solenoid valve will open and redirect the water out of the truck to the bulkhead connections on the same side as the push buttons. The water will pass through the flowmeter on the way out and help to ensure that each tanker is getting a standard size that will be included in the batch report.

The goal for reporting is to have a twenty-four hour (24-hour) water quality report on a chart in order to indicate that the water is within the established water quality parameters for the duration of the time that the system is deployed. It is also preferred to have batch reports for every load of water going through the system and out to the trucks that include the quality and quantity of water. The past reports from each day will be prepared and be available to clients at the end of each job.

### BRIEF DESCRIPTION OF THE DRAWINGS/FIGURES

The foregoing summary, as well as any detailed description of the preferred embodiments, is better understood when read in conjunction with the drawings and figures contained herein. For the purpose of illustrating the invention, the drawings and figures show certain preferred embodiments. It is understood, however, that the invention is not limited to the specific methods and devices disclosed in such drawings or figures.

FIG. 1 depicts an aerial view of a preferred embodiment of a water monitoring system of the present invention, attachably connected to both a storage tank and a truck.

FIG. 2 depicts a diagrammatic illustration of a preferred embodiment of a water monitoring system of the present invention.

FIG. 3 depicts a diagrammatic illustration of a preferred embodiment of a water monitoring system of the present invention, wherein a water sample is received within said system from a truck, moves and processes through said system, and then exits said system to a storage tank.

FIG. 4 depicts a diagrammatic illustration of a preferred embodiment of a water monitoring system of the present invention, wherein a water sample is received within said system from a storage tank, moves and processes through said system, and then exits said system to a truck.

FIG. 5 depicts a diagrammatic illustration of a preferred embodiment of a water monitoring system of the present invention, wherein a water sample is received within said system from a storage tank, moves and processes through said system for recirculation, and then exits said system back into said storage tank.

FIG. 6 depicts a front end view of a preferred embodiment of a control panel of a water monitoring system of the present invention.

### DETAILED DESCRIPTION OF A PREFERRED EMBODIMENT

A water monitoring system 100 of the present invention comprises a plurality of features that allow the system 100 to be able to receive and store substantially large amounts of water for a variety of different purposes and applications, including, but not limited to, supporting facilities with downed water systems and providing temporary water staging and distribution systems for areas affected by natural disasters. In this regard, the water monitoring system 100 of the present invention is able to receive water from alternate locations into said system 100, pump and circulate the water through said system 100, analyze the water to determine its characteristics, treat the water if necessary, and then store the water until it is needed.

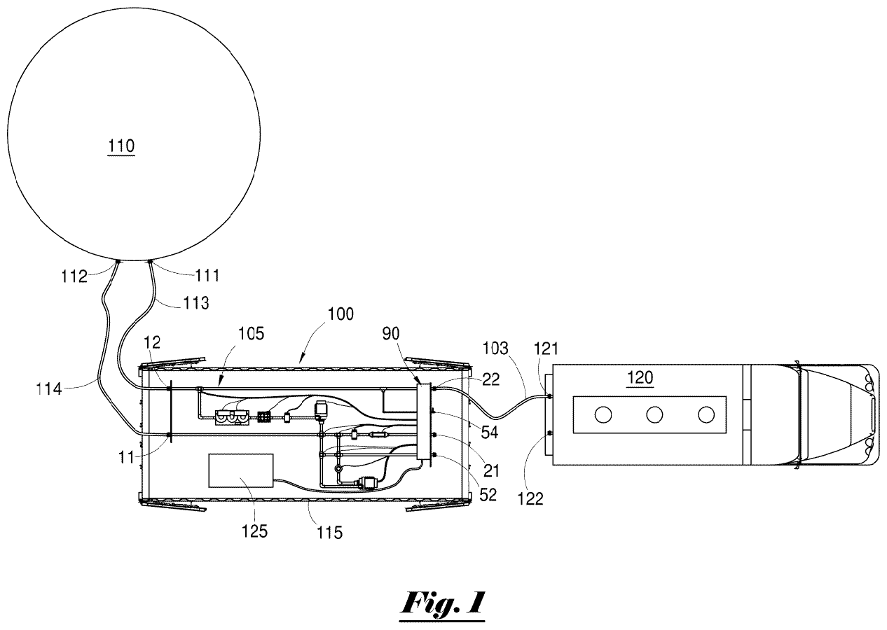

Referring to the drawings, FIG. 1 depicts an aerial view of a remote water monitoring system or unit 100 of the present invention, wherein said water monitoring system 100 is maintained within a container 115 that houses the entire system 100 and all of its component parts and features. Said water monitoring system 100 is attachably connected to both a truck 120, or any other similar delivery container, and at least one storage tank 110 each by way of a hose connection. Said truck 120 is able to drive up to said container 115 and attachably connect a hose 103 to both the truck 120 and the container 115. In addition, a separate hose line 114 is attachably connected to both said container 115 and said storage tank 110, and a separate hose line 113 is also attachably connected to both said container 115 and said storage tank 110. Depending on the particular application needed, truck 120 can either be delivering water to said system 100 in order to be stored in the storage tank 110, or the truck 120 can be hooking up to the system 100 in order to receive stored water and then deliver the water to a remote location.

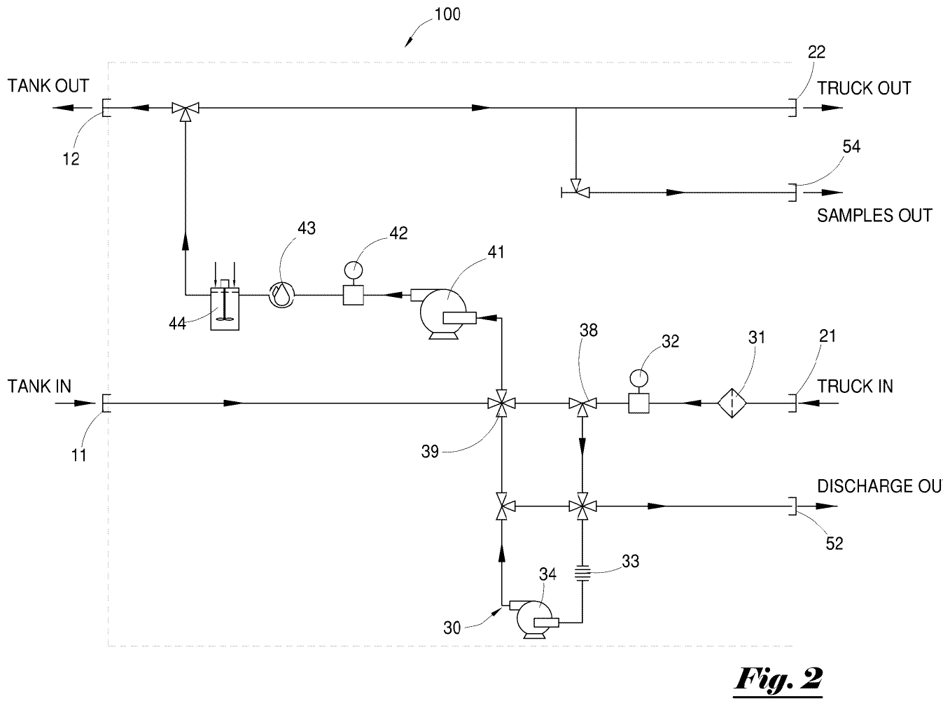

FIG. 2 depicts a schematic illustration of a water monitoring system 100 of the present invention, generally comprising multiple flow paths depending on the desired function, including, but not limited to, recirculation and monitoring water quality of water that is being stored in storage tank 110, filling storage tank 110 with a water sample provided from an alternate location and being delivered by truck 120, or filling truck 120 from water that has been stored in storage tank 110 to be delivered off site.

In order to achieve its intended functionality, the water monitoring system 100 of the present invention generally comprises a steel skid, a frame member 105, and a plurality of fittings and hose connections. By way of illustration, but not limited, said frame can be manufactured from either a stainless-steel material or a PVC material, or a combination of each, or any other similar material exhibiting like characteristics. In addition, said fittings and hose connections are manufactured from a substantially flexible material in order to maintain flexibility during transport.

It is to be noted that all materials used in the water monitoring system of the present invention must be NSF-61, or any other similar regulatory body, approved materials. The National Sanitation Foundation (NSF)'s standards help keep potable water in safe, drinkable conditions for the public. The NSF-61 certification signals that a product is safe to be installed in a potable water system for public use or consumption and is verified by a third-party.

Within said frame member 105, the present invention further comprises at least one pump, a plurality of valves, at least one probe (or sensor), at least one flowmeter, and a plurality of hoses. Further, in a preferred embodiment, the present invention comprises an external control panel and a variety of sensors, wherein said sensors communicate to a programmable logic controller (PLC) that analyzes the data provided from the sensors and then further determines any treating that needs to be done to the water supply based on the data collected.

It is to be noted that while the system 100 describes and details at least one probe and at least one pump, the number of probes or sensors and pumps can vary depending on the size and particular application of the system. As such, additional probes may be needed for additional monitoring throughout different lines within the system. Moreover, the number and size of pumps can vary, wherein one system may require one relatively larger pump, and an alternative system may require two relatively smaller pumps used in conjunction with each other. For purposes of this application, the system 100 herein will be described as using one substantially larger pump 41.

Although not depicted in the drawings, the water monitoring system 100 of the present invention comprises a flushing mechanism that clears and cleans the system 100 after contaminated water has been in the system 100. The flushing mechanism recirculates a disinfectant through the entirety of the system 100 in order to remove any remaining contaminants, and then pumps the disinfectant out through the discharge port. The flushing mechanism can then pump additional water and flush the water through the system 100 to ensure full removal of any contaminants or disinfectant.

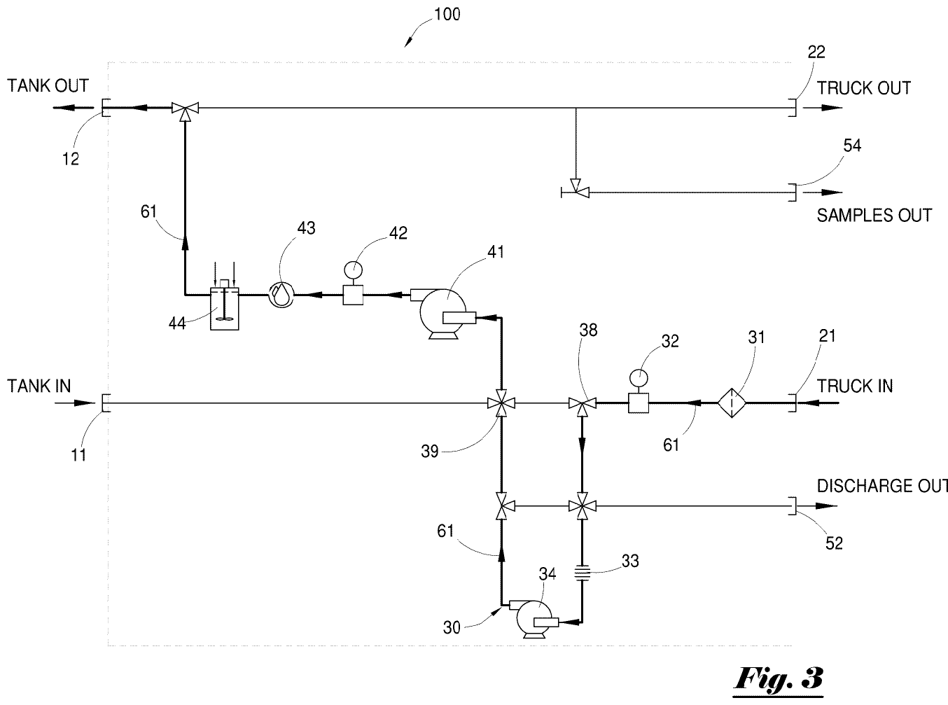

FIG. 3 depicts a preferred embodiment of a water monitoring system 100 of the present invention, wherein storage tank 110 is being filled with water that has been delivered from an alternate location via truck 120. As such, the water sample 61 is received within said system 100 from an in-line connection 21 of truck 120, is pumped through said system 100, and then exits said system through an out-line connection 12 to storage tank 110.

When filling a storage tank 110 from a truck 120, truck 120 pulls up to the water monitoring system 100 of the present invention and a driver then attachably connects a hose 103 from the truck 120 to a bulkhead connection 21 on the system 100. The driver then presses a push button 91 on an external panel 90 of the unit 100. Initially, the system 100 takes in a pre-determined amount of water, typically approximately ten (10) to fifteen (15) gallons of the delivered water, and immediately discharges this water back out of the system through a discharge outlet 52. This initial water discharge is due to the fact that any sediment and other debris from the truck will typically be settled at a bottom location of a truck 120 near the hose connection 121. As such, it is recommended to discard and discharge of this initial water intake.

Next, prior to the remaining water being pumped through the entire system 100 and into the storage tank 110, a small sample of the water is tested and validated through an intake, or validation, loop 30. In a preferred embodiment, if a quality validation process is needed first, the source water is hooked up to the intake stream by way of a hose connection 103 from the truck 120. The operator will then press a button 91 on the external control panel 90 in order to start the initial intake. The PLC will then activate a pump 34 in order to take in a predetermined sample amount of water into an intake loop 30. Water 61 will pass through an initial filter 31 to eliminate any remaining sediment or debris. By way of illustration, but not limitation, said filter comprises an in-line “Y” strainer, or any other similar filter exhibiting like characteristics (such as, an in-line cartridge filter, a basket strainer, a membrane cartridge filter, a carbon cartridge filter, a bag filter, or an ultra violet water purifier). The water 61 will then pass through a flowmeter 32 to measure the amount and volume of water going through the system 100. The system 100 will then recirculate the water 61 within the intake loop 30 until a probe 33 can obtain its data and its readings and then relay the readings to the PLC. The water 61 will continue to circulate through the intake loop 30 until there is determined to be stability in the readings obtained by the probes 33 and relayed by the PLC, wherein the readings are to be below a certain pre-determined variability.

Once the PLC has received the various readings and determined if the water is of an acceptable quality, it will operate a four-way valve 39 to release the acceptable water 61 into the remaining part of the system 100 and the intake process will continue. If the water is determined to be of unacceptable quality, an alarm will alert the operator, and then the unacceptable water will be diverted to a waste, or discharge, stream and then be discarded through the discharge port 52.

If the intake loop 30 determines that the water sample is of an acceptable quality, the remainder of the water from the truck will then be sent through the system, but the water will be able to bypass the intake loop 30 since the initial quality of the water has already been determined from the sample. The pump 41 on the main line will then pump the remaining water into the system, through a primary flowmeter 42 that will record and measure the volume of water being received through the system in gallons per minute, through an additional test probe 43 to further establish and confirm if the water quality is acceptable, and if so, then deposit the water 61 through the out port 12 and into the storage tank 110.

If the test probe 43 determines that the water characteristics are not up to a pre-determined standard, a dosing mechanism, or pump 44, will be able to treat the water with the appropriate additive (such as, for example, chlorine, phosphate, etc.) in order to bring the water quality up to standard. The dosing amount will depend on the volume or amount of water that is in the system 100, as measured and reported by the flowmeter 42. After the water has been dosed and treated, it will then be deposited through the out port 12 and into the storage tank 110.

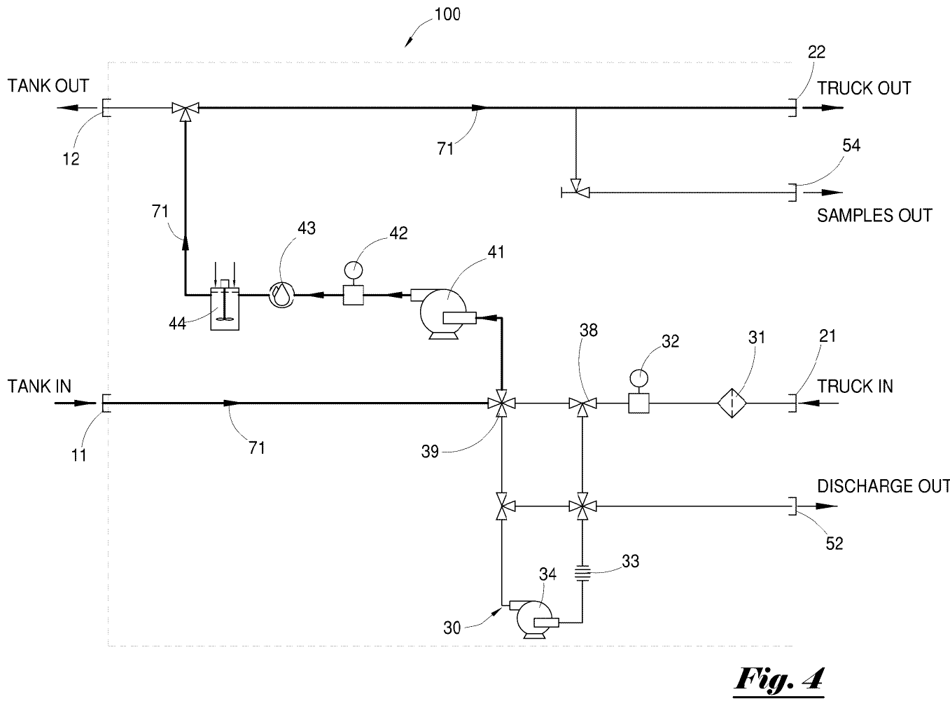

FIG. 4 depicts a preferred embodiment of a water monitoring system 100 of the present invention, wherein a water sample 71 is received within said system 100 from an intake connection 11 of storage tank 100, is pumped through said system 100, and then exits said system 100 to truck 120 to be delivered to a remote location. When filling a truck 120 from a tank 110, a driver pulls up to the water monitoring system 100 and the driver will attachably connect a hose 103 to the unit 100, press a button 91 on the external control panel 90 to fill the truck 120 and then wait for the truck 120 to be filled. Water 71 will be pumped from the storage tank 110 in through the intake line 11 and into the system 100. Water 71 passes through a pump 41 and will then pass through a flowmeter 42 and then a test probe 43 in order to determine the water quality of the water 71 that has been stored. If the test probe 43 establishes and confirms that the water quality meets the accepted standards, the pump 41 will continue to run, but after the probes, the 3-way solenoid valve 38 will open and redirect the water 71 out of the system 100 through the out line 22, to the bulkhead connections and into the truck 120. Water 71 will pass through the flowmeter on the way out to measure the water volume and to help ensure that each truck 120 is getting a standard amount of water that will be included in the batch report.

If the test probe 43 determines that the water characteristics are not up to a pre-determined standard, a dosing mechanism, or pump 44, will be able to treat the water with the appropriate additive (such as, for example, chlorine, phosphate, etc.) in order to bring the water quality up to standard. The dosing amount will depend on the volume or amount of water that is in the system 100, as measured and reported by the flowmeter 42. After the water 71 has been dosed and treated, it will then be pumped out of the system 100 and deposited into the truck 120.

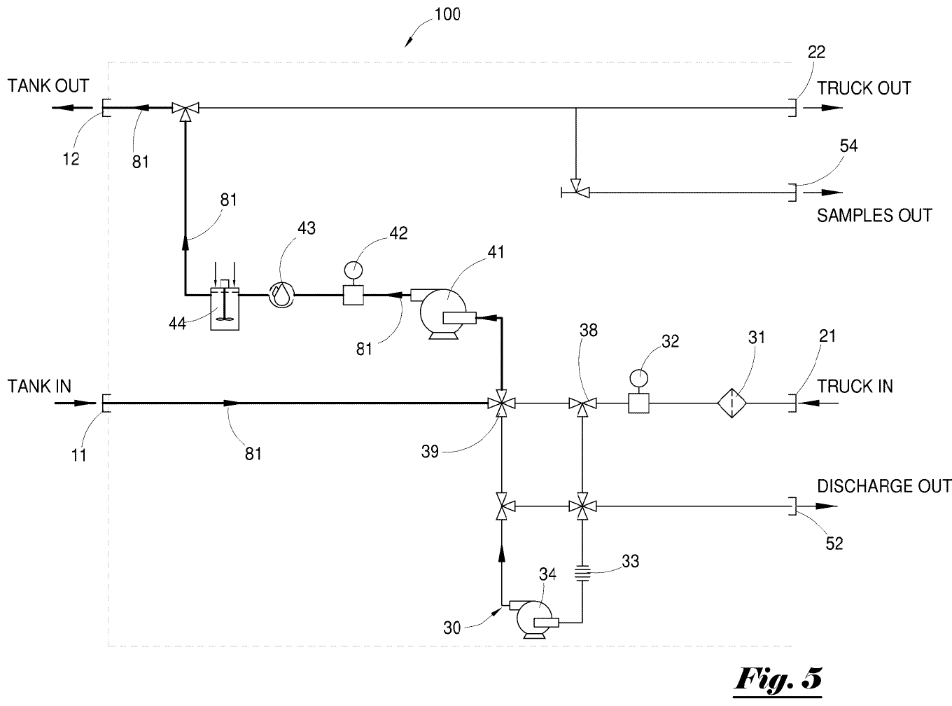

FIG. 5 depicts a preferred embodiment of water monitoring system 100 of the present invention, wherein a water sample 81 is received within said system 100 from a storage tank 110, is pumped through said system 100 for recirculation, and then exits said system 100 back into said storage tank 110. When recirculating and monitoring stored water, the water 81 from a storage tank 110 is pumped into the system 100 through an intake line 11. The water 81 passes through a four-way valve 39 in a closed position, through a pump 41, through a flowmeter 42, water then passes over a test probe 43, through a dosing pump 44, through a 3-way closed valve 38, and then pumped back out of the system 100 through out line 12 and back into the storage tank 110.

As water 81 is recirculating and being pumped through the system 100, the PLC communicates with a plurality of sensors. The sensors provide real time monitoring of a variety of different characteristics, including, but not limited to, free and total chlorine, conductivity, pH, ORP and temperature. The system 100 is able to use the sensors and the dosing pump 44 on the water stored in the storage tank 110 in order to maintain continuous dosing and to keep the water within the established parameters.

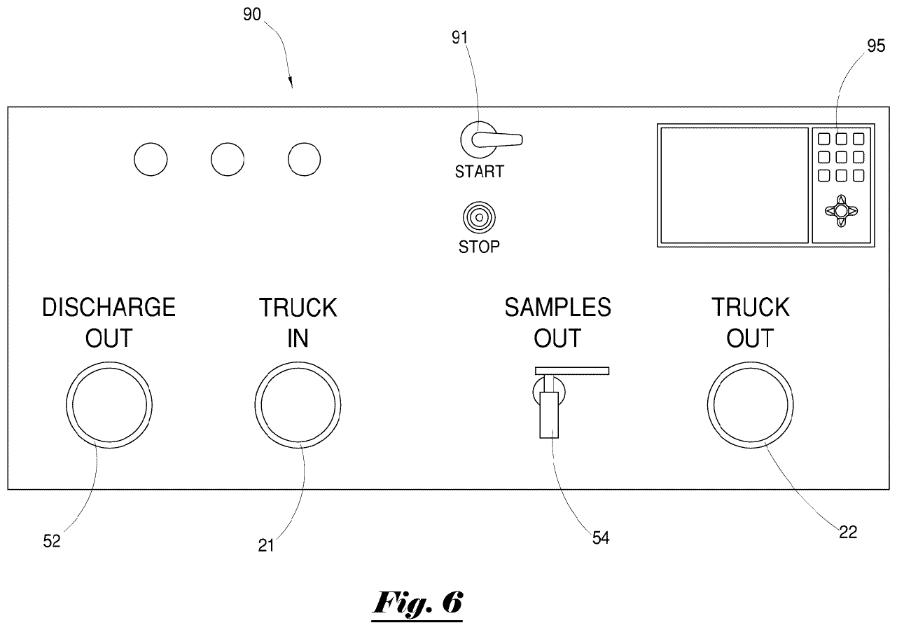

FIG. 6 depicts a front end view of a preferred embodiment of a control panel 90 of water monitoring system 100 of the present invention. The control panel 90 comprises a first connection 21 and a second connection 22, wherein said first connection 21 is for a hose attachment to pump water from truck 120 into an in-line of the system 100, and said second connection 22 is for a hose attachment to pump water out of an out-line of the system 100 and into truck 120. Additionally, said control panel 90 comprises a discharge out connection 52 and a samples out connection 54. In addition, said control panel 90 has a start switch 91 and a stop button 92 that allow the system 100 to be turned on and turned off, respectively. Said control panel 90 further comprises a display screen 95 for the user/operator. The system 100 is connected to a power source 125, such as, for example, a generator, as depicted in FIG. 1, which provides the power to the system 100 when it is turned on and started.

The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention.

What is claimed:

1. An apparatus for monitoring water quality remotely, comprising:
a. a water monitoring unit, wherein said water monitoring unit further comprises:
i. a frame member, wherein said frame member further comprises at least one in-line, at least one out-line, at least one filter, at least one pump, at least one sensor, and a plurality of valves;
ii. a control panel, wherein said control panel comprises a first connection attachment to said in-line of said frame member, a second connection attachment to said out-line of said frame member, a start switch, and a stop button;
b. a power source, wherein said power source is attachably connected to said control panel in order to provide power to said water monitoring unit;
c. at least one storage tank, wherein said storage tank is attachably connected to said frame member; and
d. a delivery container, wherein said delivery container is attachably connected to said control panel.
